(19) United States
(12) Patent Application Publication
(10) Pub. No.: US 2019/0183851 A1
(43) Pub. Date: Jun. 20, 2019

(54) USE OF 3,4,7-TRIHYDROXYISOFLAVONE OR 3-METHOXYDAIDZEIN IN PREPARATION OF DRUG FOR INHIBITING PLATELET AGGREGATION AND THROMBOSIS

(71) Applicant: Kunming Institute of Zoology, Chinese Academy of Sciences, Kunming (CN)

(72) Inventors: Ren Lai, Kunming (CN); Chuanbin Shen, Kunming (CN); Qiumin Lv, Kunming (CN); Ming Liu, Kunming (CN)

(73) Assignee: Kunming Institute of Zoology, Chinese Academy of Sciences, Kunming (CN)

(21) Appl. No.: 16/325,595

(22) PCT Filed: May 3, 2017
(86) PCT No.: PCT/CN2017/082826

§ 371 Date: Feb. 14, 2019

(30) Foreign Application Priority Data
Feb. 9, 2017 (CN) 201710070868.2

(51) Int. Cl. A61K 31/352; A61P 7/02; A61K 9/00

(52) U.S. Cl. CPC A61K 31/352; A61K 9/0053; A61P 7/02

(57) ABSTRACT

A use of 3,4,7-trihydroxyisoflavone or 3-methoxydaidzein is provided for implementation in preparation of a drug for inhibiting platelet aggregation. The 3,4,7-trihydroxyisoflavone or 3-methoxydaidzein has a significant inhibition effect on platelet aggregation, and bleeding test conducted with these embodiments show that all of the mice administrated with 3,4,7-trihydroxyisoflavone and 3-methoxydaidzein did not show any significant hemorrhagic activity, while the control group, i.e., the group administrated with the same concentration of clopidogrel showed very significant hemorrhagic activity. Thus, the use of such a drug of the present invention is useful in inhibiting platelet aggregation, and the use of such a drug can reduce the risk of bleeding, is safe for use, and expands the clinical and medical applications thereof.

### CROSS-REFERENCE TO RELATED APPLICATIONS

This application is a national phase entry of, and claims priority to, International Application No. PCT/CN2017/082826, filed May 3, 2017, which claims priority to CN 201710070868.2, filed Feb. 9, 2017. The above-mentioned patent applications are incorporated herein by reference in their entireties.

### TECHNICAL FIELD

The present invention relates to biomedicine, and more particularly relates to use of 3,4,7-trihydroxyisoflavone or 3-methoxydaidzein in preparation of a drug.

### BACKGROUND

A thrombus is a small blood clot formed by the bloodstream on the surface of an exfoliated or repaired site on an endovascular surface of a cardiovascular system. In a physiological process of coagulation, the thrombus is the final product of a cascade reaction of a blood coagulation system. It consists mainly of deposited platelets, insoluble fibrin, accumulated white blood cells and red blood cells. Platelet aggregation is one of the initial steps in a hemostasia process and is one of the important factors for thrombosis. The thrombosis in a blood vessel can impede flowing of the bloodstream, and even lead to complete occlusion of the blood vessel. The exfoliation and detachment of the thrombus from the inner wall of the blood vessel can easily form a wandering-type embolism. The cardiovascular and cerebrovascular diseases caused by this include myocardial infarction, cerebral thrombosis, arterial thromboembolism and venous thromboembolism. The thrombosis is a multi-factor varying process in which genetic and environmental factors interact and influence each other. The thrombosis has diverse onset forms, is often repetitive, and has a comprehensive incidence rate which is the highest among various diseases. Also, in recent years the incidence rate of it has been gradually increased, with persons of younger age defining the onset time of it. It is a serious threat to human health and is one of the focuses and hot spots in the contemporary medical research and drug research and development.

At present, clinically the drugs used for preventing and treating a thrombotic disease are mainly anticoagulant drugs, thrombolytic drugs and anti-platelet aggregation drugs, but common anti-thrombotic drugs such as aspirin and clopidogrel often produce side effects such as reduction of the pharmaceutical effect, gastrointestinal irritation and granulocytopenia. Additionally, aspirin, clopidogrel and the like drugs can also cause an increased risk of bleeding, which seriously affects the clinical use and the life health of a patient.

Therefore, it would be desirable to provide a use of 3,4,7-trihydroxyisoflavone or 3-methoxydaidzein in inhibiting platelet aggregation, where the use of such a drug can reduce the risk of bleeding, is safe for use, and expands the clinical and medical uses.

### SUMMARY

To achieve the above purpose, the present invention provides the following technical solutions:

In one embodiment of the invention, provided is use of 3,4,7-trihydroxyisoflavone or 3-methoxydaidzein in preparation of a drug for inhibiting platelet aggregation, where the 3,4,7-trihydroxyisoflavone has a structure shown in formula I, and 3-methoxydaidzein has a structure shown in formula II.

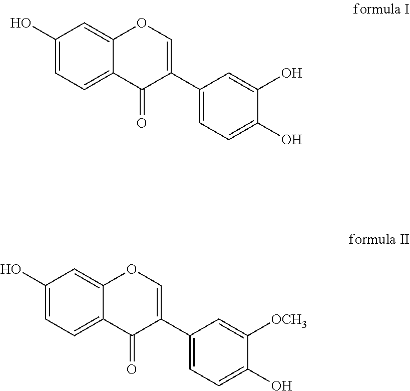 [SMILES: O=c1c(-c2ccc(O)c(O)c2)coc2cc(O)ccc12 ; COc1cc(-c2coc3cc(O)ccc3c2=O)ccc1O]

In one aspect, the platelet aggregation is generated as induced by collagen and adenosine diphosphate. For example, the dosage form of the drug is an oral preparation.

In another aspect, the administration dosage of 3,4,7-trihydroxyisoflavone depending on patients of different body weights is ≥10 μmol/kg.

In a further aspect, the administration dosage of 3-methoxydaidzein depending on patients of different body weights is ≥100 μmol/kg.

In another embodiment according to the invention, provided is use of 3,4,7-trihydroxyisoflavone or 3-methoxydaidzein in preparation of a drug for preventing and/or treating thrombosis, where the structural formulas of 3,4,7-trihydroxyisoflavone and 3-methoxydaidzein are shown by the structures in the use according to the formula diagrams above.

In one aspect, the thrombus is caused by platelet aggregation.

In another aspect, the dosage form of the drug is an oral preparation.

In a further aspect, the administration dosage of 3,4,7-trihydroxyisoflavone depending on patients of different body weights is ≥100 μmol/kg.

In yet another aspect, the administration dosage of 3-methoxydaidzein depending on patients of different body weights is ≥300 μmol/kg.

The various embodiments of the present invention provide use of 3,4,7-trihydroxyisoflavone or 3-methoxydaidzein in preparation of a drug for inhibiting platelet aggregation, where the 3,4,7-trihydroxyisoflavone has a structure shown in formula I, and 3-methoxydaidzein has a structure shown in formula II. 3,4,7-trihydroxyisoflavone or 3-methoxydaidzein has a significant inhibition effect on platelet aggregation, and meanwhile the bleeding test conducted in the embodiments shows that all of the mice administrated with 3,4,7-trihydroxyisoflavone and 3-methoxydaidzein did not show any significant hemorrhagic activity, while the control group, i.e., the group administrated with the same concentration of clopidogrel showed very significant hemorrhagic activity, which indicates that the present invention provides use of 3,4,7-trihydroxyisoflavone or 3-methoxydaidzein in inhibiting platelet aggregation, and the use of such a drug can reduce the risk of bleeding, is safe for use, and expands the clinical and medical uses.

### BRIEF DESCRIPTION OF THE DRAWINGS

Various additional features and advantages of the invention will become more apparent to those of ordinary skill in the art upon review of the following detailed description of one or more illustrative embodiments taken in conjunction with the accompanying drawings. The accompanying drawings, which are incorporated in and constitute a part of this specification, illustrate one or more embodiments of the invention and, together with the general description given above and the detailed description given below, explain the one or more embodiments of the invention.

FIG. 1 is a graphical plot showing the inhibition effect of 3,4,7-trihydroxyisoflavone on collagen-induced platelet aggregation according to an Embodiment 1 of the invention.

FIG. 2 is a graphical plot showing the inhibition effect of 3-methoxydaidzein on collagen-induced platelet aggregation in the Embodiment 1.

FIG. 3 is a graphical plot showing the inhibition effect of 3,4,7-trihydroxyisoflavone on ADP-induced mouse-tail thrombus in the Embodiment 1.

FIG. 4 is a graphical plot showing the inhibition effect of 3-methoxydaidzein on ADP-induced mouse-tail thrombus in the Embodiment 1.

FIG. 5 includes a series of photographic images showing the inhibition effect of 3,4,7-trihydroxyisoflavone and 3-methoxydaidzein on carrageenan-induced mouse-tail thrombus in an Embodiment 2 of the invention.

FIG. 6 is a graphical plot showing the changing situations of the inhibition effect of 3,4,7-trihydroxyisoflavone on carrageenan-induced mouse-tail thrombus in the Embodiment 2.

FIG. 7 is a graphical plot showing the changing situations of the inhibition effect of 3-methoxydaidzein on carrageenan-induced mouse-tail thrombus in the Embodiment 2.

FIG. 8 is a graphical plot showing the effect of 80 mg/kg 3,4,7-trihydroxyisoflavone and clopidogrel on the tail bleeding time of the mouse in an Embodiment 3 of the invention.

FIG. 9 is a graphical plot showing the effect of 80 mg/kg 3-methoxydaidzein and clopidogrel on the tail bleeding time of the mouse in the Embodiment 3.

### DETAILED DESCRIPTION

The following clearly and completely describes the technical solutions in the embodiments of the present invention with reference to the accompanying drawings in the embodiments of the present invention. To make objectives, features, and advantages of the present invention clearer, the following describes embodiments of the present invention in more detail with reference to accompanying drawings and specific implementations.

The present invention provides, in some embodiments, a use of 3,4,7-trihydroxyisoflavone or 3-methoxydaidzein in preparation of a drug for inhibiting platelet aggregation, where the 3,4,7-trihydroxyisoflavone has a structure shown in formula I, and 3-methoxydaidzein has a structure shown in formula II.

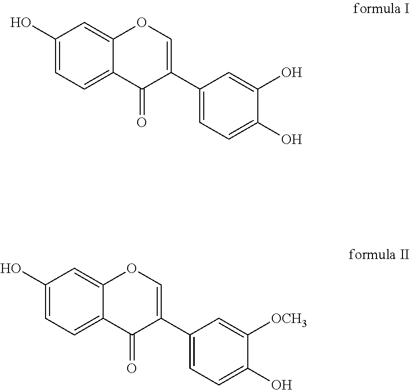 [SMILES: O=c1c(-c2ccc(O)c(O)c2)coc2cc(O)ccc12 ; COc1cc(-c2coc3cc(O)ccc3c2=O)ccc1O]

The platelet aggregation is preferably generated as induced by collagen. The induction concentration of the collagen is preferably 0.5-0.8 μg/mL, and more preferably 0.6 μg/mL. The induction concentration of the ADP is preferably 50-100 μmol/L, and more preferably 70 μmol/L.

The dosage form of the drug is preferably an oral preparation.

The administration dosage of 3,4,7-trihydroxyisoflavone for patients of different body weights is preferably ≥10 μmol/kg, more preferably from 20 μmol/kg-100 μmol/kg, and most preferably 60 μmol/kg. That is, the molar weight of the administrated drug per kg patient is ≥10 μmol/kg, where the molar weight of the administrated drug is determined depending on patients of different weights. There is no specific limitation on the source of 3,4,7-trihydroxyisoflavone, and a source of 3,4,7-trihydroxyisoflavone well known to those skilled in the art may be used. In the embodiments of the present invention, 3,4,7-trihydroxyisoflavone is purchased from Toronto Research Chemicals (North York, Canada).

The administration dosage of 3-methoxydaidzein for patients of different body weights is preferably ≥100 μmol/kg, more preferably from 120 μmol/kg-300 μmol/kg, and most preferably 200 μmol/kg. That is, the molar weight of the administrated drug per kg patient is ≥100 μmol/kg, where the molar weight of the administrated drug is determined depending on patients of different masses. There is no specific limitation on the source of 3-methoxydaidzein, and a source of 3-methoxydaidzein well known to those skilled in the art may be used. In the embodiments of the present invention, 3-methoxydaidzein is purchased from Shanghai Yuanye.

The oral preparation further includes a pharmaceutically-acceptable adjuvant. The adjuvant includes a solvent, a propellant, a solubilizer, a co-solvent, an emulsifier, a colorant, a binder, a disintegrant, a filler, a lubricant, a wetting agent, an osmotic-pressure regulator, a stabilizer, a glidant, a flavoring agent, a preservative, a suspending agent, a coating material, an aromatizer, an anti-adhesive agent, a chelating agent, a penetration enhancer, a pH adjuster, a buffering agent, a plasticizer, a surfactant, a foaming agent, a defoaming agent, a thickening agent, an inclusion agent, a humectant, an absorbing agent, a diluent, a flocculant and a deflocculant, a filter aid, a release retardant, and the like.

The administration frequency of the oral preparation is once a day, with 3 to 7 days being a course of treatment.

In further embodiments, the present invention provides the use of 3,4,7-trihydroxyisoflavone and 3-methoxydaidzein in preparation of a drug for preventing and/or treating thrombosis; and the structural formulas of 3,4,7-trihydroxyisoflavone and 3-methoxydaidzein are shown by the structures in the diagrams provided above.

The thrombus is preferably caused by platelet aggregation.

The dosage form of the drug is preferably an oral preparation.

The administration dosage of 3,4,7-trihydroxyisoflavone for patients of different body weights is preferably ≥100 μmol/kg, more preferably from 150 μmol/kg-400 μmol/kg, and most preferably 250 μmol/kg. That is, the molar weight of the administrated drug per kg patient is ≥100 μmol/kg, where the molar weight of the administrated drug is determined depending on patients of different masses.

The administration dosage of 3-methoxydaidzein for patients of different body weights is preferably ≥300 μmol/kg, more preferably from 300 μmol/kg-500 μmol/kg, and most preferably 400 μmol/kg. That is, the molar weight of the administrated drug per kg patient is ≥300 μmol/kg, where the molar weight of the administrated drug is determined depending on patients of different masses.

The oral preparation further includes a pharmaceutically-acceptable adjuvant. The adjuvant includes one or more of a solvent, a propellant, a solubilizer, a co-solvent, an emulsifier, a colorant, a binder, a disintegrant, a filler, a lubricant, a wetting agent, an osmotic-pressure regulator, a stabilizer, a glidant, a flavoring agent, a preservative, a suspending agent, a coating material, an aromatizer, an anti-adhesive agent, a chelating agent, a penetration enhancer, a pH adjuster, a buffering agent, a plasticizer, a surfactant, a foaming agent, a defoaming agent, a thickening agent, an inclusion agent, a humectant, an absorbing agent, a diluent, a flocculant and a deflocculant, a filter aid and a release retardant.

In further embodiments, the present invention further provides a method for preparing the oral preparation including the following mass percentages of components: 5-20% of an active ingredient, 2-10% of a disintegrant, 0.2-2% of a lubricant, 0.1-1.5% of a glidant and 0-0.5% of an additive, with the balance being a filler, where the components are mixed to prepare an oral preparation; and the active ingredient is 3,4,7-trihydroxyisoflavone or 3-methoxydaidzein.

The administration frequency of the oral preparation is once a day, with 3 to 7 days being a course of treatment.

The use of 3,4,7-trihydroxyisoflavone or 3-methoxydaidzein provided by the present invention in the preparation of a drug for inhibiting platelet aggregation and thrombus is described in detail below with reference to Embodiments, but they are not to be construed as limiting the claimed scope of the present invention.

### Embodiment 1

Healthy human platelets were diluted with plasma to 2.5×10⁸ platelets per mL. 300 μL of plasma-rich platelets was taken, added into different concentrations of samples at 37° C. and maintained at this temperature for 5 min, then added with 70 μmol/L ADP to induce aggregation, and an aggregation curve within 5 min was plotted on a platelet aggregation instrument. Platelet aggregation without sample treatment was used as a control.

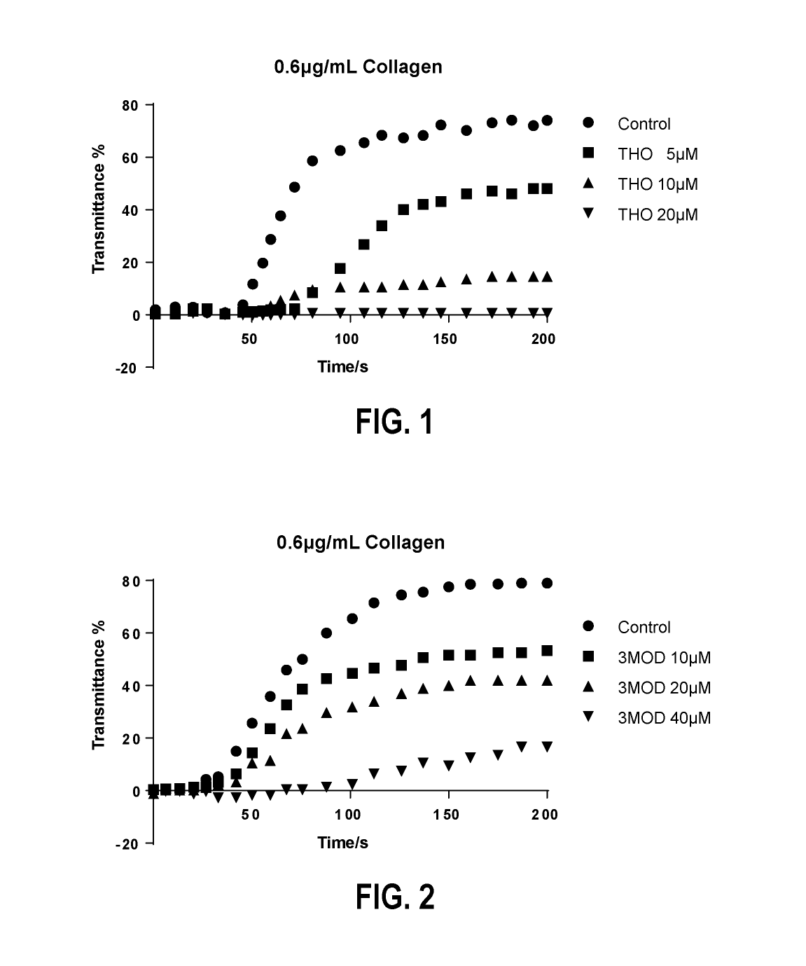

The collagen-induced platelet aggregation was detected by using washed human platelets. As shown in FIGS. 1 and 2, 3,4,7-trihydroxyisoflavone and its analogs inhibited platelet aggregation induced by 0.6 μg/mL collagen in a gradient-dependent manner. 20 μM of 3-methoxydaidzein could inhibit about 50% of platelet aggregation, while 20 μM of 3,4,7-trihydroxyisoflavone could completely inhibit platelet aggregation. The platelet washing method was as follows: a. 1.4 mL platelets (concentrated platelet plasma) were poured into each 1.5 mL EP tube, and centrifuged at 400 g under room temperature for 5 min, and the supernatant was discarded; b. the platelets in each tube were resuspended with 0.5 mL of a tyrode A solution, and two tubes of resuspended platelets were combined into a new 1.5 mL EP tube, mixed well and centrifuged at 400 g under room temperature for 5 min; c. the platelets in each tube were resuspended with 1 mL of the tyrode A solution, transferred into a new EP tube, and centrifuged at room temperature at 400 g under room temperature for 5 min; d. the platelets in each tube were resuspended with 1 mL of a tyrode B solution, and the platelets were adjusted to an appropriate concentration with the tyrode B solution.

| | | Tyrode A solution | | Tyrode B solution | |
|---|---|---|---|---|---|
| Tyrode stock | | (PH 6.5): | | (PH 7.35-7.45): | |
| solution (1 L): | | 50 mL stock solution | | 50 mL stock solution | |
| NaHCO₃ | 1 g | BSA | 0.175 g | Glucose | 0.05 g |
| NaCl | 8 g | 0.5M EDTA | 250 μl | | |
| NaH₂PO₄ | 0.05 g | Glucose | 0.05 g | | |
| KCl | 0.2 g | | | | |

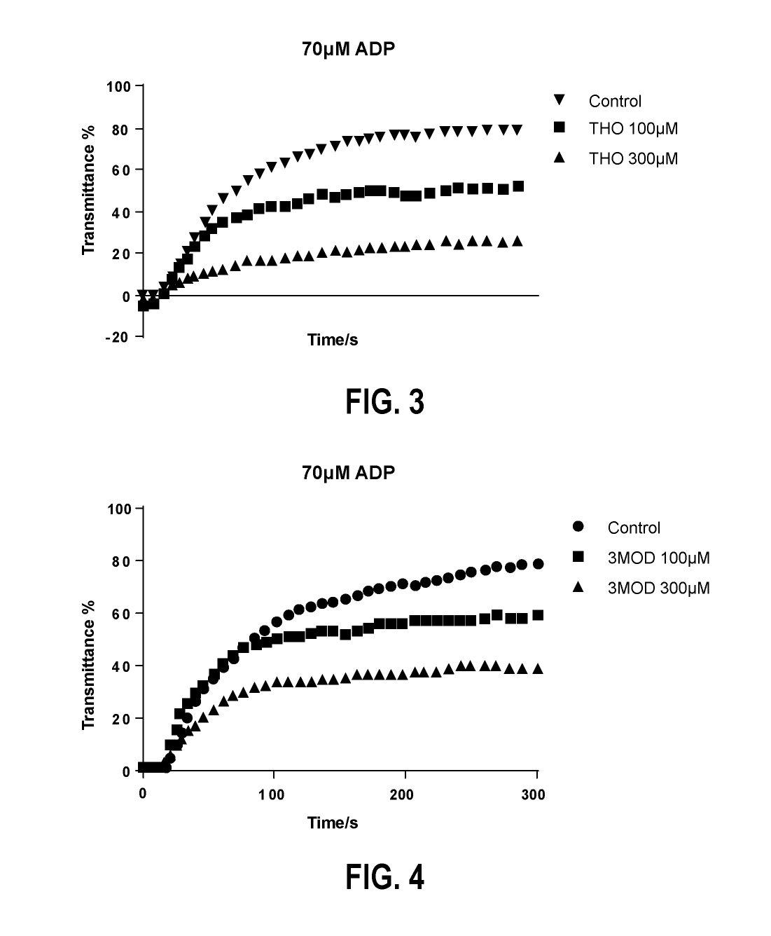

As shown in FIGS. 3 and 4, the diagrams respectively represented that 3,4,7-trihydroxyisoflavone (3′,4′,7-trihydroxyisoflavone) and 3-methoxydaidzein (3′-methoxydaidzein) inhibited ADP-induced platelet aggregation in PRP in a gradient-dependent manner. 300 μM of 3-methoxydaidzein could inhibit about 50% of platelet aggregation, while 100 μM of 3,4,7-trihydroxyisoflavone could inhibit about 50% of platelet aggregation.

### Embodiment 2

This embodiment relates to a carrageenan-induced mouse-tail thrombosis model.

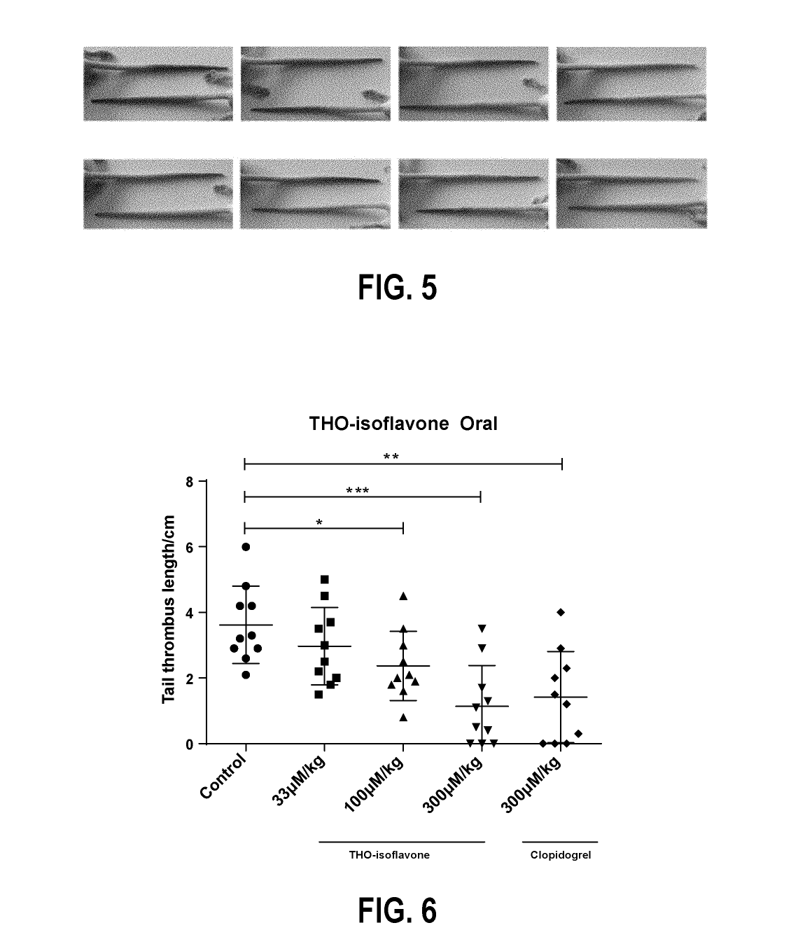

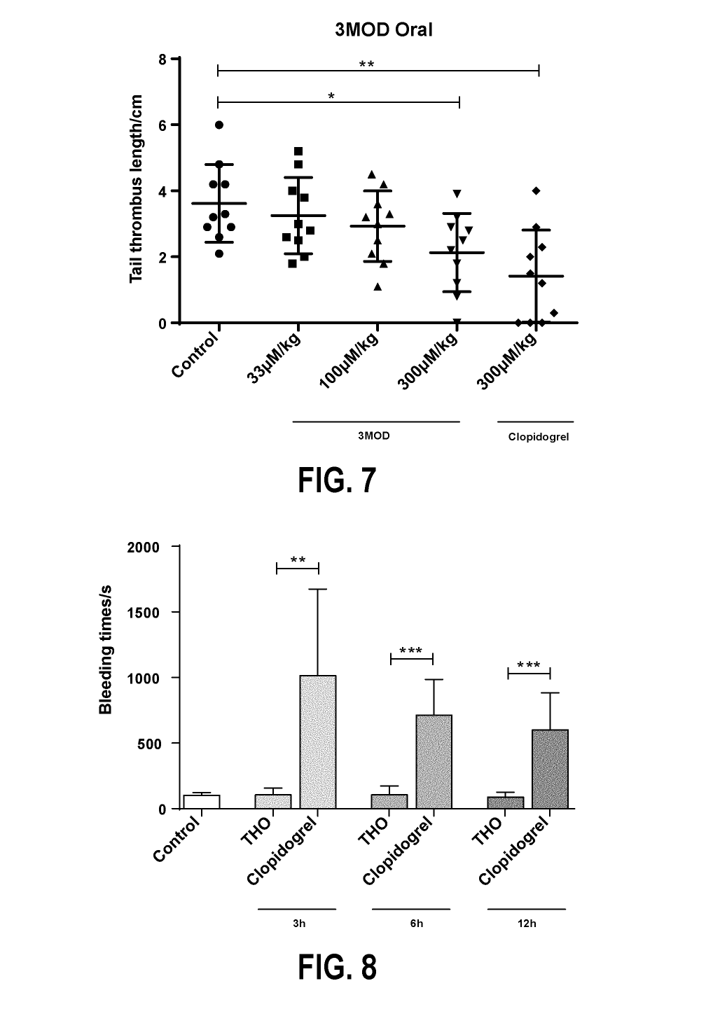

The experiment animals, male Kunming mice, each with a body weight of 20-25 g, were randomly divided into groups (n=10) after fed for one week. The first group was a DMSO control group, the sample groups were gavaged with 3,4,7-trihydroxyisoflavone and 3-methoxydaidzein at concentrations respectively of 33, 100 and 300 μmol/kg, and the positive control was gavaged with 300 μmol/kg clopidogrel. 30 min after intragastric administration, carrageenan (type I, Sigma) was injected intraperitoneally into the mice at a dose of 40 mg/kg. Since the rate of thrombosis was >90% at a low temperature, the feeding temperature was 18° C. After 24 h, the mean length of thrombosis was determined according to the color change of the tail skin. The rate of thrombus inhibition by oral administration of 3,4,7-trihydroxyisoflavone and 3-methoxydaidzein was increased along with the increase of concentration. Compared with the negative control group, both oral administration of 300 μmol/kg of 3-methoxydaidzein and oral administration of 100 μmol/kg of 3,4,7-trihydroxyisoflavone could significantly inhibit thrombosis in a mouse tail, where the average thrombus length of the group gavaged with 300 μmol/kg of 3,4,7-trihydroxyisoflavone was shorter than that of the group gavaged with 300 μmol/kg of clopidogrel (FIGS. 5-7).

### Embodiment 3

This embodiment relates to an evaluation of bleeding risk of 3,4,7-trihydroxyisoflavone and 3-methoxydaidzein.

Each group included 8 male C57BL/6 (18-20 g). The test sample group and the control group were administered by gavage. After 3, 6 and 12 hours of administration, the mice were placed into a self-made special holder to make the tails pass through a trimmed 1 mL pipette tip. 5 mm of the tail tip was cut off with a sterilized razor blade, and the mouse tail was immediately immersed into normal saline at 37° C. The bleeding time of the tail was observed by using the bloodstream stop time as an indicator.

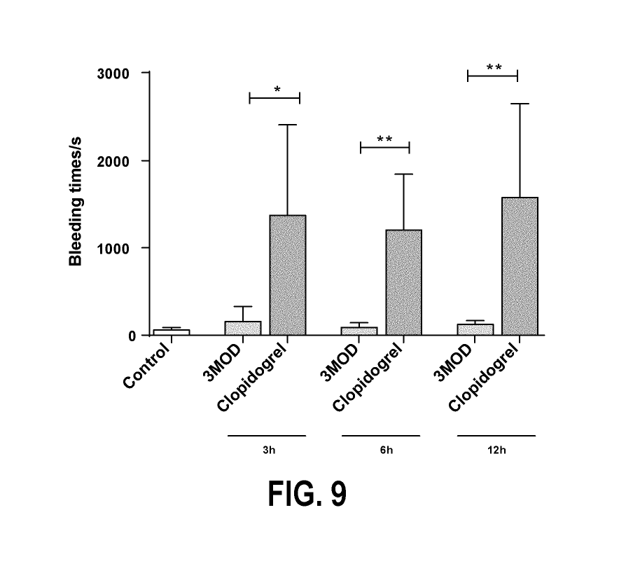

The results were shown in FIG. 8, the group administrated with 3,4,7-trihydroxyisoflavone at the dose of 80 mg/kg did not show any significant bleeding activity at various time points compared with the non-administrated group, while the group administrated with clopidogrel at the same concentration increased bleeding significantly. The results were shown in FIG. 9, the group administrated with 3-methoxydaidzein at the dose of 80 mg/kg did not show any significant bleeding activity at various time points compared with the non-administrated group, while the group administrated with clopidogrel at the same concentration increased bleeding significantly.

The above description of the embodiment is only for helping to understand the method of the present invention and its core idea. It should be noted that, several improvements and modifications may be made by persons of ordinary skill in the art without departing from the principle of the present invention, and these improvements and modifications should also be considered within the protection scope of the present invention. Various modifications to these embodiments are clear to persons skilled in the art, and the generic principles defined herein may be practiced in other embodiments without departing from the spirit or scope of the invention. Thus, the present invention is not limited to the embodiments shown herein but falls within the widest scope consistent with the principles and novel features disclosed herein.

The embodiments described above are only descriptions of preferred embodiments of the present invention and are not intended to limit the scope of the present invention. Various variations and modifications can be made to the technical solution of the present invention by those of ordinary skill in the art, without departing from the design and spirit of the present invention. The variations and modifications should all fall within the claimed scope defined by the claims of the present invention.

What is claimed is:

1.-10. (canceled)

11. A use of 3,4,7-trihydroxyisoflavone or 3-methoxydaidzein in preparation of a drug for inhibiting platelet aggregation, wherein the 3,4,7-trihydroxyisoflavone has a structure shown in formula I, and 3-methoxydaidzein has a structure shown in formula II:

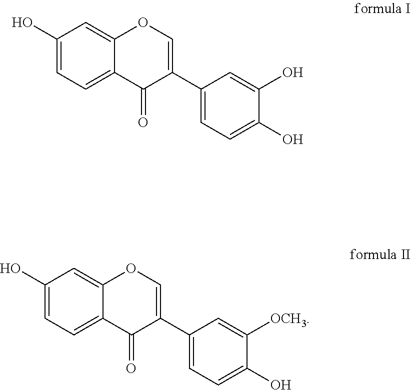 [SMILES: O=c1c(-c2ccc(O)c(O)c2)coc2cc(O)ccc12 ; COc1cc(-c2coc3cc(O)ccc3c2=O)ccc1O]

12. The use of claim 11, wherein a dosage form of the drug is an oral preparation.

13. The use of claim 12, wherein an administration dosage of 3,4,7-trihydroxyisoflavone depending on patients of different body weights is ≥10 μmol/kg.

14. The use of claim 12, wherein an administration dosage of 3-methoxydaidzein depending on patients of different body weights is ≥100 μmol/kg.

15. The use of claim 11, wherein the platelet aggregation is generated as induced by collagen and adenosine diphosphate.

16. The use of claim 15, wherein a dosage form of the drug is an oral preparation.

17. The use of claim 16, wherein an administration dosage of 3,4,7-trihydroxyisoflavone depending on patients of different body weights is ≥10 μmol/kg.

18. The use of claim 16, wherein an administration dosage of 3-methoxydaidzein depending on patients of different body weights is ≥100 μmol/kg.

19. A use of 3,4,7-trihydroxyisoflavone or 3-methoxydaidzein in preparation of a drug for preventing and/or treating thrombosis, wherein the 3,4,7-trihydroxyisoflavone has a structure shown in formula I, and 3-methoxydaidzein has a structure shown in formula II:

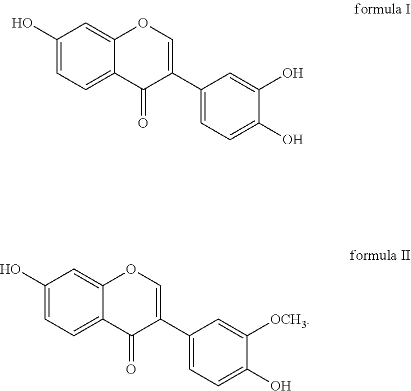 [SMILES: O=c1c(-c2ccc(O)c(O)c2)coc2cc(O)ccc12 ; COc1cc(-c2coc3cc(O)ccc3c2=O)ccc1O]

20. The use of claim 19, wherein a dosage form of the drug is an oral preparation.

21. The use of claim 20, wherein an administration dosage of 3,4,7-trihydroxyisoflavone depending on patients of different body weights is ≥100 μmol/kg.

22. The use of claim 20, wherein an administration dosage of 3-methoxydaidzein depending on patients of different body weights is ≥300 μmol/kg.

23. The use of claim 19, wherein the thrombus is caused by platelet aggregation.

24. The use of claim 23, wherein a dosage form of the drug is an oral preparation.

25. The use of claim 24, wherein an administration dosage of 3,4,7-trihydroxyisoflavone depending on patients of different body weights is ≥100 μmol/kg.

26. The use of claim 24, wherein an administration dosage of 3-methoxydaidzein depending on patients of different body weights is ≥300 μmol/kg.
